(19) United States
(12) Patent Application Publication
(10) Pub. No.: US 2010/0122519 A1
(43) Pub. Date: May 20, 2010

(54) ULTRA-LOW SULFUR FUEL AND METHOD FOR REDUCED CONTRAIL FORMATION

(76) Inventors: Alan Epstein, Lexington, MA (US); Richard C. Miake-Lye, Lexington, MA (US)

Correspondence Address: CARLSON, GASKEY & OLDS/PRATT & WHITNEY, 400 WEST MAPLE ROAD, SUITE 350, BIRMINGHAM, MI, 48009, US

(21) Appl. No.: 12/614,640

(22) Filed: Nov. 9, 2009

Related U.S. Application Data
(60) Provisional application No. 61/114,486, filed on Nov. 14, 2008.

(51) Int. Cl. F02C 3/20; C10L 1/04; C07C 9/00

(52) U.S. Cl. 60/39.461; 208/16; 585/16

(57) ABSTRACT

A method of managing contrail formation of a gas turbine engine includes delivering an ultra-low sulfur fuel to a combustor of a gas turbine engine to limit an amount of sulfur byproduct produced in an exhaust of the gas turbine engine.

### CROSS REFERENCE TO RELATED APPLICATION

This application claims priority to U.S. Provisional No. 61/114,486, filed Nov. 14, 2008.

### BACKGROUND OF THE INVENTION

This disclosure relates to contrails made by the exhaust from aircraft engines. Hot exhaust from aircraft engines causes visible trails of condensed water known as contrails under known temperature and humidity conditions. For instance, the hot exhaust mixes with the cooler, moist surrounding air and causes condensation/precipitation of water as minute droplets or ice crystals. There is speculation that contrails contribute to changing the Earth's climate by containing outgoing radiation, either directly or as amplified by stimulating the formation of clouds, similar to greenhouse gases. Additionally, contrail formation may be a concern for aircraft operators that wish to minimize visible detection.

In some instances, an aircraft may avoid producing contrails by avoiding flying into contrail-forming conditions. However, depending on the aircraft, flight pattern, or other circumstances, it is not always possible to avoid contrail-forming conditions. In addition, such contrail avoidance flight patterns may be longer and/or require more total fuel to be burned, which would increase cost and produce more CO₂ with the effect of increasing rather than decreasing global warming.

Additives to the fuel or to the exhaust may be used to reduce contrail formation. For instance, the additives influence the size of the condensed water droplets or ice crystals. Droplets or ice crystals of certain sizes may not be visible. However, problems with using additives include the cost of the additives (effectively increasing the cost of the fuel which is already a significant concern in the aviation industry), the weight associated with the additives, and any reduction in engine life or performance caused by the additives.

### SUMMARY OF THE INVENTION

An example method of managing contrail formation of a gas turbine engine includes delivering an ultra-low sulfur fuel to a combustor of a gas turbine engine to limit an amount of sulfur byproduct produced in an exhaust of the gas turbine engine.

Another example method of managing contrail formation of a gas turbine engine includes establishing a critical threshold of sulfur byproducts in an exhaust of a gas turbine engine such that below a critical threshold, the exhaust substantially reduces contrail formation when the gas turbine engine is flying in contrail-forming conditions.

An example ultra-low sulfur aviation fuel composition includes a concentration of sulfur that is less than one part per million.

### BRIEF DESCRIPTION OF THE DRAWINGS

The various features and advantages of the disclosed examples will become apparent to those skilled in the art from the following detailed description. The drawings that accompany the detailed description can be briefly described as follows.

FIG. 1 illustrates an example gas turbine engine utilizing an ultra-low sulfur fuel while flying in contrail-forming conditions.

### DETAILED DESCRIPTION OF THE PREFERRED EMBODIMENT

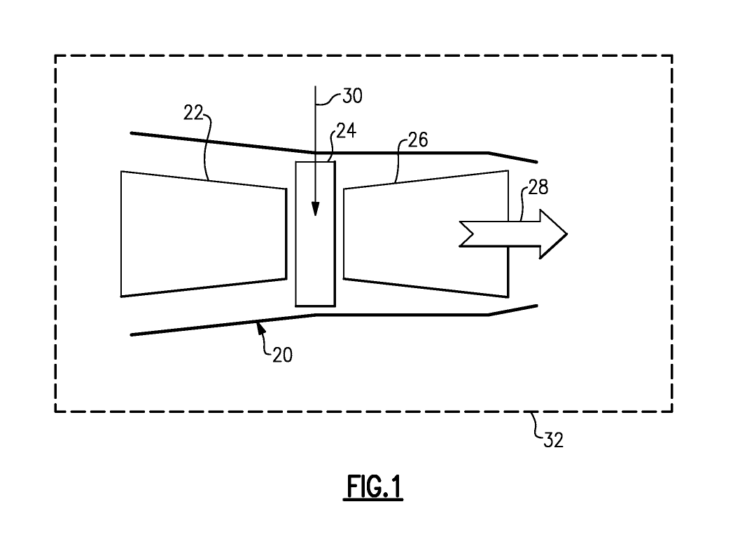

FIG. 1 illustrates selected portions of an example gas turbine engine 20 that may be employed on an aircraft. In this example, the gas turbine engine 20 includes a compressor section 22, a combustor 24 for receiving compressed air from the compressor section 22, and a turbine section 26 for receiving an exhaust 28 from the combustor 24. It is to be understood that there are various types of gas turbine engines, many of which can benefit from the disclosed examples, which are not limited to the disclosed design.

The gas turbine engine 20 utilizes an ultra-low sulfur fuel 30 to manage contrail formation from the gas turbine engine 20 while flying through contrail-forming conditions 32. Typical aviation fuels include 30-3000 parts per million of sulfur. However, the ultra-low sulfur fuel 30 of the disclosed example includes a concentration of sulfur that is less than about one part per million to limit an amount of sulfur byproduct produced in the exhaust 28. In further examples, the ultra-low sulfur fuel 30 may have a concentration of sulfur that is less than 300 parts per billion or concentration that is below detectable limits (i.e., nominally zero). The sulfur byproducts (e.g., including SO₃) may be in the form of particles, compounds, or other exhaust matter emitted in the exhaust 28.

Sulfur byproducts may act as nucleation sites for condensation of water under contrail-forming conditions. For instance, sulfur byproducts are highly hydroscopic compared to carbon or other particles in an exhaust. The sulfur byproducts, including any sulfur byproduct associated with emitted soot particles, attract water vapor molecules more readily than other types of particles in the exhaust. The sulfur byproduct may rapidly accumulate water and form droplets that lead to contrails. The amount of sulfur byproduct in the exhaust 28 is limited by utilizing the ultra-low sulfur fuel 30. The ultra-low sulfur fuel 30 thereby limits or eliminates contrail formation because there is limited sulfur byproduct in the exhaust 28 to support nucleation and water droplet formation.

Reducing or eliminating contrail formation using the ultra-low sulfur fuel 30 provides the benefits of reduced concern of aviation-induced climate change, no increase in effective fuel cost or aircraft weight from additives, no impact on fuel consumption, and reduction of particulate emission.

Desired concentrations of sulfur in the ultra-low sulfur fuel 30 for reducing or eliminating contrail formation may be predetermined. For instance a critical threshold of sulfur byproducts in the exhaust 28 may be established such that below the critical threshold, the exhaust 28 substantially reduces contrail formation when the gas turbine engine 20 is flying in the contrail-forming condition 32. In one example, the reduction in contrail formation may correspond to the degree of visibility of a contrail, average water droplet size or other parameter for judging contrail formation.

The ultra-low sulfur fuel 30 may be produced using any of a variety of methods. In one example, the ultra-low sulfur fuel 30 may be a Fischer-Tropsch synthetic paraffin fuel that is made without adding sulfur or sulfur-containing additives. In this regard, the ultra-low sulfur fuel 30 may include a concentration of sulfur that is close to zero or undetectable.

In another example, the ultra-low sulfur fuel 30 may be produced by removing sulfur from an existing type of aviation fuel. The existing fuel may be a synthetic fuel or a petroleum-based fuel, for example. For instance, a sulfur-removing device could be used to remove sulfur from the existing fuel.

Regardless of the source of the ultra-low sulfur fuel 30, one concern with using the ultra-low sulfur fuel 30 might be sulfur contamination from existing fuel supply chains. For instance, aviation fuels are typically transported through a supply chain that may handle a variety of different types of fuels. Residual amounts of one type of fuel may remain in the supply chain and intermix with subsequently transported fuels. Normally, the intermixing is insignificant and does not influence engine performance. However, mixing even a small amount of a sulfur-containing fuel with the ultra-low sulfur fuel 30 may increase the sulfur concentration above a desired/threshold concentration for contrail formation. Similarly, the gas turbine engine 20 or the aircraft fuel system may include residual sulfur-containing fuel. Therefore, the supply chain, the aircraft, and gas turbine engine 20 may require cleaning to limit contamination.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from the essence of this disclosure.

What is claimed is:

1. A method of managing contrail formation of a gas turbine engine, comprising:
delivering an ultra-low sulfur fuel to a combustor of a gas turbine engine to limit an amount of sulfur byproduct produced in an exhaust of the gas turbine engine.

2. The method as recited in claim 1, wherein the gas turbine engine is flying in contrail-forming conditions.

3. The method as recited in claim 1, wherein a concentration of sulfur in the ultra-low sulfur fuel is less than one parts per million.

4. The method as recited in claim 1, wherein a concentration of sulfur in the ultra-low sulfur fuel is less than 300 parts per billion.

5. An ultra-low sulfur aviation fuel composition comprising:
an aviation fuel having a concentration of sulfur that is less than one part per million.

6. The ultra-low sulfur aviation fuel as recited in claim 5, wherein the concentration of sulfur is the less than 300 parts per billion.

7. The ultra-low sulfur aviation fuel as recited in claim 5, wherein the concentration of sulfur is nominally zero.

8. The ultra-low sulfur aviation fuel as recited in claim 5, wherein the aviation fuel is a synthetic paraffin fuel.

9. The ultra-low sulfur aviation fuel as recited in claim 5, wherein the aviation fuel is a petroleum-based fuel.

10. A method of managing contrail formation of a gas turbine engine, comprising:
establishing a critical threshold of sulfur byproducts in an exhaust of a gas turbine engine such that below the critical threshold the exhaust substantially reduces contrail formation when the gas turbine engine is flying in contrail-forming conditions.
